(19) United States
(12) Patent Application Publication
(10) Pub. No.: US 2014/0277282 A1
(43) Pub. Date: Sep. 18, 2014

(54) USE OF COMPOUND ACTION POTENTIALS TO AUTOMATICALLY ADJUST NEUROSTIMULATION THERAPY IN RESPONSE TO POSTURAL CHANGES OF PATIENT

(71) Applicant: BOSTON SCIENTIFIC NEUROMODULATION CORPORATION, Valencia, CA (US)

(72) Inventor: Kristen Jaax, Santa Clarita, CA (US)

(73) Assignee: BOSTON SCIENTIFIC NEUROMODULATION CORPORATION, Valencia, CA (US)

(21) Appl. No.: 14/201,882

(22) Filed: Mar. 9, 2014

Related U.S. Application Data
(60) Provisional application No. 61/801,490, filed on Mar. 15, 2013.
(60) Provisional application No. 61/808,263, filed on Apr. 4, 2013.

(51) Int. Cl. A61N 1/36

(52) U.S. Cl. CPC A61N 1/36139; A61N 1/3615; A61N 1/36185
USPC 607/59

(57) ABSTRACT

A neurostimulation system and method of treating a patient. Electrical stimulation energy is delivered to a target tissue site in accordance with a stimulation parameter to treat the patient and evoke at least one compound action potential (CAP) in a population of neurons. A magnitude of the evoked CAP is measured. A function of the measured evoked CAP magnitude(s) is compared to a threshold value. The stimulation parameter is adjusted based on the comparison.

### RELATED APPLICATIONS DATA

The present application claims the benefit under 35 U.S.C. §119 to U.S. Provisional Application Ser. No. 61/801,490, filed Mar. 15, 2013 and U.S. Provisional Application Ser. No. 61/808,263, filed Apr. 4, 2013, which applications are all incorporated herein by reference in their entirety.

### FIELD OF THE INVENTION

The present invention relates to tissue stimulation systems, and more particularly, to a system and a method for automatically adjusting the amplitude of stimulation based on postural changes in lead position.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCM) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Furthermore, Functional Electrical Stimulation (FES) systems, such as the Freehand system by NeuroControl (Cleveland, Ohio), have been applied to restore some functionality to paralyzed extremities in spinal cord injury patients. Further, in recent investigations, Peripheral Nerve Stimulation (PNS) systems have demonstrated efficacy in the treatment of chronic pain syndromes and incontinence, and a number of additional applications are currently under investigation. For example, Occipital Nerve Stimulation (ONS), in which leads are implanted in the tissue over the occipital nerves, has shown promise as a treatment for various headaches, including migraine headaches, cluster headaches, and cervicogenic headaches.

These implantable neurostimulation systems typically include one or more electrode carrying neurostimulation leads, which are implanted at the desired stimulation site, and a neurostimulator (e.g., an implantable pulse generator (IPG)) implanted remotely from the stimulation site, but coupled either directly to the neurostimulation lead(s) or indirectly to the neurostimulation lead(s) via a lead extension. Thus, electrical pulses can be delivered from the neurostimulator to the neurostimulation leads to stimulate the tissue and provide the desired efficacious therapy to the patient. The neurostimulation system may further comprise a handheld patient programmer in the form of a remote control (RC) to remotely instruct the neurostimulator to generate electrical stimulation pulses in accordance with selected stimulation parameters.

A typical stimulation parameter set may include the amplitude, duration, and rate of the electrical stimulation energy (e.g., electrical stimulation pulses), as well as the electrode combination, which identifies the electrodes functioning as anodes or cathodes. The RC may, itself, be programmed by a clinician, for example, by using a clinician's programmer (CP), which typically includes a general purpose computer, such as a laptop, with a programming software package installed thereon. Typically, the RC can only control the neurostimulator in a limited manner (e.g., by only selecting a program or adjusting the pulse amplitude or pulse width), whereas the CP can be used to control all of the stimulation parameters, including which electrodes are cathodes or anodes. Thus, in accordance with the stimulation parameters programmed by an external control device, such as an RC or a CP, electrical pulses can be delivered from the neurostimulator to the stimulation electrode(s) to stimulate or activate a volume of tissue in accordance with a set of stimulation parameters and provide the desired efficacious therapy to the patient.

Typically, a set of stimulation parameters is configured by a clinician so that appropriate stimulation energy is delivered to the volume of tissue that is targeted for the intended therapeutic benefit (e.g., treatment of pain) without causing undesirable side effects. However, movements or postural changes of the patient may necessitate adjustment of the stimulation parameters. This is because the neurostimulation leads tend to migrate relative to themselves, as well as relative to the targeted tissue to be stimulated as the patient moves or undergoes postural changes. For example, in SCM, a neurostimulation lead can move within the epidural space in which it is implanted. Also, the movements or postural changes of the patient can create problems in ONS applications, where turning the head side to side either forces the lead closer to the nerve or can displace the lead farther away from the nerve, depending on the direction the head is turned. Clinically, such variations of distance between the neurostimulation lead and the targeted neural tissue caused by the patent's movement and/or postural changes can result in over- or under-stimulation of the targeted tissue or even stimulate non-targeted tissue.

Thus, there is a need to provide an efficient and effective way to dynamically adjust the stimulation in response to the patient's activities and postural changes.

### SUMMARY OF THE INVENTION

In accordance with one aspect of the present inventions, a neurostimulation system is provided. The neurostimulation system comprises a plurality of electrical terminals respectively configured for being electrically coupled to a plurality of electrodes implanted within a neural tissue, stimulation output circuitry coupled to the plurality of electrical terminals, monitoring circuitry coupled to the plurality of electrical terminals, and memory configured for storing a threshold value.

The neurostimulation system further comprises controller/processor circuitry configured for prompting the stimulation output circuitry to deliver electrical stimulation energy to a first set of the electrodes in accordance with a stimulation parameter capable of treating a patient, and prompting the stimulation output circuitry to evoke at least one compound action potential (CAP) in a population of neurons in the neural tissue.

The controller/processor circuitry is further configured for prompting the monitoring circuitry to measure a magnitude of the at least one evoked CAP (eCAP) at a second set of the electrodes. The neurostimulation system may further comprise one or more neurostimulation leads carrying the first set of electrodes and the second set of electrodes. The controller/processor circuitry is further configured for comparing a function of the measured magnitude(s) of the eCAP(s) to the threshold value, and adjusting the stimulation parameter based on the comparison. In one embodiment, a plurality of CAPs are evoked and measured, in which case, the function may be an average of the eCAPs.

The stimulation parameter that is adjusted may be an electrical pulse parameter, in which case, the controller/processor circuitry may be configured for adjusting the electrical pulse parameter to vary an intensity level of the delivered electrical stimulation energy. For example, the controller/processor circuitry may be configured for increasing the electrical pulse parameter if the function of the at least one measured eCAP magnitude is below the threshold value, and for decreasing the electrical pulse parameter if the function of the at least one measured eCAP magnitude is above the threshold value. The controller/processor circuitry may be configured for comparing the function of the measured eCAP magnitude(s) to a first threshold value and a second threshold value greater than the first threshold value. In this case, the controller/processor circuitry may be configured for adjusting the electrical pulse parameter to increase the intensity level if the function of the measured eCAP magnitude(s) is below the first threshold value, adjusting the electrical pulse parameter to decrease the intensity level if the function of the measured eCAP magnitude(s) is above the second threshold value, and not adjusting the electrical pulse parameter if the function of the eCAP magnitude(s) is between the first threshold value and the second threshold value.

The stimulation parameter that is adjusted may be an electrode combination (e.g., a fractionalized electrode combination), in which case, the controller/processor circuitry may be configured for adjusting the electrode combination to displace a resulting electrical field relative to the neural tissue. For example, the controller/processor circuitry may be configured for comparing the function of the measured eCAP magnitude(s) to first threshold value and a second threshold value greater than the first threshold value. In this case, the controller/processor circuitry may be configured for adjusting the electrode combination to displace the resulting electrical field in a first direction if the function of the measured eCAP magnitude(s) is below the first threshold value, adjusting the electrode combination to displace the resulting electrical field in a second direction opposite to the first direction if the function of the measured eCAP magnitude(s) is above the second threshold value, and not adjusting the electrode combination if the function of the eCAP magnitude(s) is between the first threshold value and the second threshold value.

In accordance with a second aspect of the present inventions, a method of treating a patient is provided. The method comprises delivering electrical stimulation energy to a target tissue site in accordance with a stimulation parameter to treat the patient, evoking at least one compound action potential (CAP) in a population of neurons, and measuring a magnitude of the evoked CAP (eCAP). The electrical stimulation energy may be delivered from a lead implanted adjacent the target tissue site, and the lead may migrate relative to the target tissue site prior to the delivery of the electrical stimulation energy to the target tissue site. The CAP(s) may be evoked in the population of neurons by delivering electrical stimulation energy to a first set of electrodes, and the eCAP magnitude(s) may be measuring at a second set of electrodes. The first set of electrodes and the second set of electrodes may be carried on a lead parallel to the population of neurons.

The controller/processor circuitry is further configured for comparing a function of the measured eCAP magnitude(s) to a threshold value (which may be indicative of a therapeutic goal), and adjusting the stimulation parameter based on the comparison. In one embodiment, a plurality of CAPs are evoked and measured, in which case, the function may be an average of the eCAPs.

In one method, the stimulation parameter is an electrical pulse parameter that is adjusted to vary an intensity level of the delivered electrical stimulation energy. For example, the electrical pulse parameter may be adjusted to increase the intensity level if the function of the measured eCAP magnitude(s) is below the threshold value, and adjusted to decrease the intensity level if the function of the measured eCAP magnitude(s) is above the threshold value. The function of the measured eCAP magnitude(s) may be compared to first threshold value and a second threshold value greater than the first threshold value. In this case, the electrical pulse parameter may be adjusted to increase the intensity level if the function of the measured eCAP magnitude(s) is below the first threshold value, adjusted to decrease the intensity level if the function of the measured eCAP magnitude(s) is above the second threshold value, and not adjusted if the function of the eCAP magnitude(s) is between the first threshold value and the second threshold value.

In another method, the stimulation parameter is an electrode combination (e.g., a fractionalized electrode combination) that is adjusted to displace a resulting electrical field relative to the target tissue site. The function of the measured eCAP magnitude(s) may be compared to first threshold value and a second threshold value greater than the first threshold value. In this case, the electrode combination may be adjusted to displace the resulting electrical field in direction away from the target tissue site if the function of the measured eCAP magnitude(s) is below the first threshold value, is adjusted to displace the resulting electrical field in a direction toward the target tissue site if the function of the measured eCAP magnitude(s) is above the second threshold value, and not adjusted if the function of the eCAP magnitude(s) is between the first threshold value and the second threshold value.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal Cord Stimulation (SCS) system constructed in accordance with one embodiment of the present inventions;

FIG. 2 is a plan view of an implantable pulse generator (IPG) and two percutaneous stimulation leads used in the SCS system of FIG. 1;

FIG. 3 is a plan view of the SCS system of FIG. 1 in use within a patient;

FIG. 4 is a schematic block diagram showing an exemplary internal component configuration of the IPG of FIG. 2.

FIG. 5 is a flow diagram illustrating one exemplary method of performing a stimulation parameter adjustment technique performed by the SCS system of FIG. 1; and

FIG. 6 is a flow diagram illustrating another exemplary method of performing a stimulation parameter adjustment technique performed by the SCS system of FIG. 1.

### DETAILED DESCRIPTION OF THE EMBODIMENTS

The description that follows relates to various aspects, embodiments, and/or specific features or sub-components of the present disclosure being provided within a Spinal Cord Stimulation (SCS) system. However, it is to be understood that, while the various aspects, embodiments, and/or specific features or sub-components of the present disclosure lend themselves well to applications in SCS, the present disclosure, in its broadest aspects, is not limited to being used for SCS or in a SCS system. Rather, the various aspects, embodiments, and/or specific features or sub-components of the present disclosure may be used with any type of implantable electrical circuitry used to stimulate any tissue. For example, the present disclosure may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc. Of course, those of ordinary skill in the art understand that the above-listed examples are merely exemplary and are not exhaustive or limiting.

Turning first to FIG. 1, an exemplary SCS system 10 adapted for automatically adjusting stimulation level by using compound action potentials is described. The SCS system 10 generally includes one or more implantable neurostimulation leads 12(1) and 12(2), an implantable pulse generator (IPG) 14, an external remote controller RC 16, a clinician's programmer (CP) 18, an External Trial Stimulator (ETS) 20, and an external charger 22.

The IPG 14 may be physically connected to the neurostimulation leads 12 via one or more percutaneous lead extensions 24. Each of the neurostimulation leads 12 may carry a plurality of electrodes 26, which may be arranged in an array. In the illustrated embodiment, the neurostimulation leads 12 are percutaneous leads, and to this end, the electrodes 26 are arranged in-line along the neurostimulation leads 12. In an alternative embodiment, however, the electrodes 26 may be arranged in a two-dimensional pattern on a single paddle lead. Also, it should be appreciated that the number of neurostimulation leads and electrodes may vary depending on the type of neurostimulation system and its application. As will be described in further detail below, the IPG 14 includes pulse generation circuitry that delivers the electrical stimulation energy in the form of an electrical pulse train to the electrode array 26 according to a set of stimulation parameters.

The ETS 20 may also be physically connected via the percutaneous lead extensions 28 and external cable 30 to the neurostimulation leads 12. The ETS 20, which may include a similar pulse generation circuitry as the IPG 14, can also deliver electrical stimulation in the form of an electrical pulse train to the electrode array 26. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, it should be understood that all functionalities of the IPG 14 described herein can also be implemented with the ETS 20 to the extent that such functionalities are not dependent on implantation of the ETS 20.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14.

The CP 18 allows a clinician to obtain detailed information regarding the stimulation parameters for programming the IPG 14 and ETS 20 in the operating room and in follow-up sessions. For instance, the CP 18 can be used to perform more in-depth analysis on the therapeutic effect of a certain stimulation parameter or a set of stimulation parameters, which may require more computing power than the RC 16 or other components implanted within the patient's body. Also, the CP 18 may lock and/or unlock various features and functionalities on IPG 14 and/or RC 16. For example, CP 18 may impose maximum and/or minimum values for each of the stimulation parameters so that it cannot be subsequently changed by IPG 14 and RC 16 beyond the limitation without the assistance of CP 18. The CP 18 may perform such functions by communicating with the RC 16 via a communication link 36, thereby indirectly communicating with the IPG 14 (and/or ETS 20). Alternatively, the CP 18 may communicate directly with the IPG 14 (and/or ETS 20) to provide, or otherwise configure, the stimulation parameters. The communication links 32, 34 and 36 may be implemented with any suitable communication technologies, including but not limited to radio frequency, infrared, electromagnetic, and/or inductive based communication links. The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 may charge the IPG 14 via a wire (not shown) or wirelessly charge the IPG 14 via a suitable wireless charging techniques. For instance, the external charger 22 may transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present.

For purposes of brevity, the details of the RC 16, CP 18, ETS 20 and external charger 22 will not be described herein. Details of exemplary embodiments of these devices are disclosed in U.S. Pat. No. 6,895,280, which is expressly incorporated herein by reference.

Referring now to FIG. 2, the external features of exemplary neurostimulation leads 12 and the IPG 14 will be briefly described. One of the neurostimulation leads 12(1) has eight electrodes 26 (labeled E1-E8), and the other neurostimulation lead 12(2) has eight electrodes 26 (labeled E9-E16). Of course, the number and shape of the leads and the electrodes may vary based on the intended application of the neurostimulation system. Further details describing the construction and method of manufacturing percutaneous neurostimulation leads are disclosed in U.S. Pat. No. 8,019,439, entitled “Lead Assembly and Method of Making Same,” and U.S. Pat. No. 7,650,184, entitled “Cylindrical Multi-Contact Electrode Lead for Neural Stimulation and Method of Making Same,” which are expressly incorporated herein by reference. In some embodiments, a surgical paddle lead can be utilized, the details of which are disclosed in U.S. Patent Publication. No. 2007/0150036 A1, entitled “Stimulator Leads and Methods for Lead Fabrication and 2012/0059446 A1 entitled Collapsible/Expandable Tubular Electrode Leads,” which is expressly incorporated herein by reference.

The IPG 14 comprises an outer case 40 for housing the electronic and other components (described in further detail below), and a connector 42 to which the proximal ends of the neurostimulation leads 12 mate in a manner that electrically couples the electrodes 26 to the electronics within the outer case 40. The outer case 40 is composed of an electrically conductive, biocompatible material, such as titanium, and forms a hermetically sealed compartment wherein the internal electronics are protected from the body tissue and fluids. In some cases, the outer case 40 may serve as an electrode.

The IPG 14 includes a pulse generation circuitry that provides electrical stimulation energy to the electrodes 26 in accordance with a set of stimulation parameters. Such parameters may include electrode combinations, which define the electrodes that are activated as anodes (positive), cathodes (negative), and turned off (zero). The stimulation parameters may further include pulse amplitude (measured in milliamps or volts depending on whether the IPG 14 supplies constant current or constant voltage to the electrodes), pulse width (measured in microseconds), pulse rate (measured in pulses per second), duty cycle (pulse width divided by cycle duration), burst rate (measured as the stimulation energy on duration X and stimulation energy off duration Y), and pulse shape.

With respect to the pulse patterns provided during operation of the system 10, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the IPG outer case 40. Electrical energy may be transmitted to the tissue in a monopolar or multipolar (for example, bipolar, tripolar and similar configurations) fashion or by any other means available.

Monopolar delivery occurs when a selected one or more of the lead electrodes 26 is activated along with the case 40 of the IPG 14, so that electrical energy is transmitted between the selected electrode 26 and outer case 40. In this setting, the electrical current has a path from the energy source contained within the IPG outer case 40 to the tissue and a sink path from the tissue to the energy source contained within the case. Monopolar delivery may also occur when one or more of the lead electrodes 26 are activated along with a large group of lead electrodes located remotely from the one or more lead electrodes 26 so as to create a monopolar effect; that is, electrical energy is conveyed from the one or more lead electrodes 26 in a relatively isotropic manner. Bipolar delivery occurs when two of the lead electrodes 26 are activated as anode and cathode, so that electrical energy is transmitted between the selected electrodes 26. Tripolar delivery occurs when three of the lead electrodes 26 are activated, two as anodes and the remaining one as a cathode, or two as cathodes and the remaining one as an anode.

The electrical energy may be delivered between electrodes as monophasic electrical energy or multiphasic electrical energy. Monophasic electrical energy includes a series of pulses that are either all positive (anodic) or all negative (cathodic). Multiphasic electrical energy includes a series of pulses that alternate between positive and negative. For example, multiphasic electrical energy may include a series of biphasic pulses, with each biphasic pulse including a cathodic (negative) stimulation pulse and an anodic (positive) recharge pulse that is generated after the stimulation pulse to prevent direct current charge transfer through the tissue, thereby avoiding electrode degradation and cell trauma.

That is, a charge is conveyed through the electrode-tissue interface via current at an electrode during a stimulation period (the length of the stimulation pulse), and then pulled back off the electrode-tissue interface via an oppositely polarized current at the same electrode during a recharge period (the length of the recharge pulse). The recharge pulse may be active, in which case, the electrical current is actively conveyed through the electrode via current or voltage sources, or the recharge pulse may be passive, in which case, the electrical current may be passively conveyed through the electrode via redistribution of the charge flowing from coupling capacitances present in the circuit.

As shown in FIG. 3, the neurostimulation leads 12 are implanted within the spinal column 46 of a patient 48. The preferred placement of the neurostimulation leads 12 is adjacent, i.e., resting near, or upon the dura, adjacent to the spinal cord area to be stimulated. The neurostimulation leads 12 will be located in a vertebral position that depends upon the location and distribution of the chronic pain. For example, if the chronic pain is in the lower back or legs, the neurostimulation leads 12 may be located in the mid- to low-thoracic region (e.g., at the T9-12 vertebral levels). Due to the lack of space near the location where the neurostimulation leads 12 exits the spinal column 46, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

Significant to the present inventions, the SCS system 10, in addition to conveying electrical stimulation energy to a target tissue site to treat the patient, is capable of analyzing measured evoked compound action potentials (eCAPs) and making appropriate adjustments to the stimulation parameters in order to maintain the conveyed electrical stimulation energy at an efficacious and comfortable level. An eCAP is the simultaneous evoking of action potentials traveling down a population of neurons. Thus, the total magnitude of the eCAP is proportional to the number of neurons that are carrying action potentials, and therefore, may function as a clinical measurement as to the intensity level (i.e., strength of the conveyed electrical stimulation energy), which is both the dose of therapy that is used to decrease the pain in the patient, and the physiological signal that causes the patient to perceive either comfortable paresthesia, painful overstimulation, or lack of stimulation. The eCAP measurement can then be used in a closed loop fashion to adjust the stimulation parameters in real-time in order to compensate for postural changes in the patient and continuously provide efficacious therapy to the patient, while avoiding overstimulation or understimulation that may otherwise result from postural changes or otherwise lead migration. With an incorrect level of stimulation energy delivered from the IPG 14, the paresthesia can be lost or cause undesired side effects to the patient.

For this reason, the SCS system 10 is configured for repeatedly evoking, measuring, and analyzing CAPs to adjust the intensity level of the delivered stimulation. The intensity of the delivered stimulation energy can be increased or decreased by adjusting one or more of the stimulation parameters, such as the pulse rate, pulse amplitude, pulse width, and pulse duty cycle. The electrode combination (e.g., selection of anode(s) and cathode(s)) is another stimulation parameter, which may be adjustable in displacing the resulting electrical field.

In the preferred embodiment, the SCS system 10 is configured for automatically adjusting one or more of stimulation parameters until the magnitude of the evoked action potentials indicates that the stimulation energy is optimally delivered to the target tissue site. In an alternative embodiment, the stimulation parameter adjustment can be performed manually by the clinician or the patient, in which case, the SCS system 10 provides suggested stimulation parameter values. The SCS system 10 may include a reference database containing a list of previous evoked action potential measurements correlated to corresponding stimulation parameter sets.

In the illustrated embodiment, the CAPs are evoked by conveying the same electrical stimulation energy used to provide therapy to the patient to a first set of electrodes 26 (which may be a single electrode or multiple electrodes grouped together) to evoke CAPs in the neural tissue adjacent these electrode(s) 26, and measuring the resulting eCAPs at a second different set of electrodes 26 (which may be a single electrode or multiple electrodes grouped together). To increase the signal-to-noise ratio, the SCS system 10 may average the magnitudes of sequentially evoked CAPs, and then use this average to adjust the set of stimulation parameters.

The SCS system 10 analyzes the measured eCAPs by comparing their magnitudes to stored threshold value(s) that are indicative of a therapeutic goal. For example, two threshold values may define the lower and upper ends of a magnitude range of eCAPs indicative of good coupling efficiency between the electrodes 26 from which the electrical stimulation energy is conveyed and the target tissue site. The measured eCAPs can be compared to these two thresholds to determine whether the set of stimulation parameters should be adjusted. In particular, if the measured eCAP drops below the lower threshold value, it may be determined that the coupling efficiency is such that the intensity level of the currently conveyed electrical stimulation energy is not high enough and/or the active electrode combination is not properly selected to provide efficacious therapy to the patient. In contrast, if the measured eCAP rises above the upper threshold value, it may be determined that the coupling efficiency is such that the intensity level of the currently conveyed electrical stimulation energy is too high and/or the active electrode combination is improperly selected, resulting in painful or otherwise uncomfortable side effects for the patient. Notably, the lower and upper threshold values may be represented by single value having a tolerance. In this case, the set of stimulation parameters may be adjusted when the magnitude of the measured eCAP deviates from the threshold value by a certain amount.

Accordingly, if the stimulation parameter to be adjusted is an electrical pulse parameter (e.g., a pulse amplitude, pulse width, or pulse rate), the electrical pulse parameter can be adjusted in a manner that increases the intensity level of the electrical stimulation energy if the magnitude of the measured eCAP is below the lower threshold value; adjusted in a manner that decreases the intensity level of the electrical stimulation energy if the magnitude of the measured eCAP is above the upper threshold value; and not adjusted at all if the magnitude of the measured eCAP is between the lower and upper threshold values.

If the stimulation parameter to be adjusted is an electrode combination (e.g., a fractionalized electrode combination), the electrode combination can be adjusted in a manner that displaces the electrical field resulting from the conveyed electrical stimulation energy in first direction if the magnitude of the measured eCAP is below the lower threshold value; adjusted in a manner that displaces the electrical field resulting from the conveyed electrical stimulation energy in second direction opposite the first direction if the magnitude of the measured eCAP is above the high threshold value; and not adjusted at all if the magnitude of the measured eCAP is between the lower and upper threshold values.

Although the SCS system 10 may instruct the IPG 14 to output the electrical stimulation energy to the electrodes 26 based on a set of stimulation parameters, not all stimulation parameters in the set need to be adjusted. This is especially true when the purpose of adjustment is to simply obtain the evoked action potential that matches the threshold value or a range of the threshold value. For example, one step size increase or decrease in the pulse amplitude may be enough to evoke the targeted eCAP. On the other hand, different combinations of stimulation parameters may need to be tried with different values. Of course, multiple iterations of parameter adjustments, stimulation using the adjusted parameters, followed by the comparison of evoked action potential characteristics to the threshold value or the threshold value range may be required to finalize the stimulation energy that provides the optimum therapy to the patient.

As will be described in further detail below, the stimulation parameter set adjustment process is entirely performed in the IPG 14. However, external programming devices, such as the RC 16 or the CP 18, may alternatively be configured to analyze the eCAP measurement and provide a new set of stimulation parameters for the IPG 14 or otherwise make suitable adjustments on one or more of the stimulation parameters for the IPG 14. In this case, the RC or CP 18 may retrieve the eCAP measurements from the IPG 14, and provide a new stimulation parameter set or a control signal (e.g., signals for adjusting the stimulation parameters) to the IPG 14.

The automatic stimulation adjustment process described above may be triggered based on various pre-defined conditions. For example, the stimulation adjustment process may be initiated immediately upon detecting that the magnitude of the evoked action potential is greater than or less than the pertinent threshold value. In some cases, however, the magnitude of the eCAP may differ due to a short-lasting postural change, an acute lead movement, or temporary impedance change at the target stimulation site by various other factors. Constantly adjusting stimulation parameters to obtain perfectly matching evoked action potential in such cases may render the SCS system 10 rather inefficient. In these cases, the SCS system 10 may automatically adjust the set of stimulation parameters upon a determination that the measured eCAP is less than or greater than the pertinent threshold value only after a predetermined time period or a predetermined number of comparisons.

In addition to the eCAP measurement, the SCS system 10 may optionally utilize various additional physiological signals measured from the patient to trigger the afore-mentioned stimulation parameter set adjustment process. To this end, the SCS system 10 may comprise various sensors (not shown) to obtain a variety of physiological information from the patient, such as patient's body activities, temperature, blood flow, electrocortigram, electroencephalogram, tissue or transcutaneous oxygen tension, glucose concentration, electrode impedance, intra/extra cellular potential or electrical current, as well as chemical species concentration, which may be monitored and analyzed via the IPG 14, RC 16, and/or CP 18. It should be noted that the sensors for measuring such extra physiological information may be integrated in the IPG 14, on the neurostimulation leads 12, and/or provided as standalone sensors.

The SCS system 10 may optionally employ a learning algorithm, such as a neural network, to optimize the stimulation parameter adjustment technique. For example, the SCS system 10 may be configured to track various types of information, including but not limited to, the difference between the eCAP measurement and the threshold value(s), the amount that the stimulation parameter(s) are adjusted, and the resulting outcome of the adjustment (i.e., closeness of the eCAP measurement to the threshold value after the adjustment). Such information may be stored and analyzed by the system 10 for future use and to minimize the number of adjustments on the stimulation parameters necessary to reach the efficacious stimulation level. For example, the SCS system 10 may determine a step size to adjust the relevant stimulation parameter. The additional physiological information described above may also be used in implementing the learning algorithm. For example, the SCS system 10 may select which stimulation parameter to adjust based on a certain sensed postural movement of the patient.

Turning next to FIG. 4, one exemplary embodiment of the IPG 14 will now be described. The IPG 14 includes stimulation output circuitry 50 configured for generating electrical stimulation energy in accordance with an electrical pulse train having a specified pulse amplitude, pulse rate, pulse width, duty cycle, burst rate, and shape under control of control logic 52 over data bus 54. The pulse rate and the duration of stimulation may be controlled by analog circuitry, or digital timer logic circuitry 56 controlling the analog circuitry, and which may have a suitable resolution, e.g., 10 μs. In alternative embodiments, a continuous modulating waveform may be generated by the stimulation output circuitry 50 in a manner described in U.S. Provisional Patent Application Ser. No. 61/646,773, entitled “System and Method for Shaped Phased Current Delivery,” which is expressly incorporated herein by reference. The stimulation energy generated by the stimulation output circuitry 50 is output via capacitors C1-C16 to electrical terminals 58 respectively corresponding to electrodes E1-E16.

In the illustrated embodiment, the stimulation output circuitry 50 may either include independently controlled current sources for providing stimulation pulses of a specified and known amperage to or from the electrical terminals 58, or independently controlled voltage sources for providing stimulation pulses of a specified and known voltage at the electrical terminals 58 or to multiplexed current or voltage sources that are then connected to the electrical terminals 58. The operation of this stimulation output circuitry 50, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference. Thus, it can be appreciated that the stimulation output circuitry 50 is capable of delivering electrical energy to the electrodes 26 via the electrical terminals 58 for the purpose of providing therapy to the patient and/or evoking CAPs in the neural tissue for the purpose of adjusting the stimulation parameters.

The IPG 14 also includes monitoring circuitry 60 for monitoring the status of various nodes or other points 62 throughout the IPG 14, e.g., power supply voltages, temperature, battery voltage, and the like. Notably, the electrodes 26 fit snugly within the epidural space of the spinal column, and because the tissue is conductive, electrical measurements can be taken from the electrodes 26 in order to determine the coupling efficiency between the respective electrode 26 and the tissue and/or to facilitate fault detection with respect to the connection between the electrodes 26 and the stimulation output circuitry 60 of the IPG 14. More significant to the present inventions, the monitoring circuitry 60 is configured to measure a magnitude of the eCAPs generated by stimulation of neural tissue via the stimulation output circuitry 50. The evoked potential measurement technique may be performed by generating an electrical field at a set of the electrodes 26, which is strong enough to depolarize the neurons adjacent the stimulating electrode beyond a threshold level, thereby inducing the firing of an eCAP that propagates along the neural fibers. Such stimulation is preferably supra-threshold, but not uncomfortable. A suitable stimulation pulse for this purpose is, for example, 4 mA for 200 μs. While a selected one or more of the electrodes 26 is activated to generate the electrical field, another set of the electrodes 26 (different from the activated electrode set) is operated to record a measurable deviation in the voltage caused by the evoked potential due to the stimulation pulse at the stimulating electrode set. To the extent that other physiological information is acquired for the purpose of triggering the stimulation parameter adjustment process, the monitoring circuitry 60 may be coupled to those sensors. If the physiological measurements are electrical, the sensors may be one or more of the electrodes 26. For other types of non-electrical physiological information, however, separate sensors may be used for appropriate measurements. Specific implementations of such optional sensors will depend on the nature of the physiological information to be measured.

The IPG 14 further includes a controller/processor circuitry in the form of a microcontroller (μC) 64 (or a processor) that controls the control logic 52 over data bus 66, and obtains status data from the monitoring circuitry 60 via data bus 68. The IPG 14 additionally controls the timer logic 56. The IPG 14 further includes memory 70 and oscillator and clock circuit 72 coupled to the microcontroller 64. The memory 70 may store various data (e.g. stimulation parameters, threshold values, etc.) and series of instructions to be executed by the microcontroller 64. The microcontroller 64, in combination with the memory 70 and oscillator and clock circuit 72, thus comprise a microprocessor system that carries out a program function in accordance with a suitable program stored in the memory 70. Alternatively, for some applications, the function provided by the microprocessor system may be carried out by a suitable state machine.

Thus, the microcontroller 64 generates the necessary control and status signals, which allow the microcontroller 64 to control the operation of the IPG 14 in accordance with a selected operating program and stimulation parameters. In controlling the operation of the IPG 14, the microcontroller 64 is able to individually generate electrical energy at the electrodes 26 using the stimulation output circuitry 50, in combination with the control logic 52 and timer logic 56, thereby allowing each electrode 26 to be paired or grouped with other electrodes 26, including the monopolar case electrode, to control the polarity, pulse amplitude, pulse rate, pulse width, and pulse duty cycle through which the electrical energy is provided. The microcontroller 64 is also capable of automatically implementing the stimulation parameter adjustment process described above.

The IPG 14 further includes an alternating current (AC) receiving coil 74 for receiving programming data (e.g., the operating program and/or stimulation parameters) from the RC 16 and/or CP 18 in an appropriate modulated carrier signal, and charging and forward telemetry circuitry 76 for demodulating the carrier signal it receives through the AC receiving coil 74 to recover the programming data, which programming data is then stored within the memory 70, or within other memory elements (not shown) distributed throughout the IPG 14.

The IPG 14 further includes a back telemetry circuitry 78 and an alternating current (AC) transmission coil 80 for sending informational data sensed through the monitoring circuitry 60 to the RC 16 and/or CP 18. The back telemetry features of the IPG 14 also allow its status to be checked. Any changes made to the current stimulation parameters are confirmed through back telemetry, thereby assuring that such changes have been correctly received and implemented within IPG 14. Moreover, upon interrogation by the RC 16 and/or CP 18, all programmable settings stored within the IPG 14 may be uploaded to the RC 16 and/or CP 18.

Notably, if the RC 16, or alternatively the CP 18, is used to perform the automated stimulation parameter adjustment technique, the measured eCAPs can be transmitted from the IPG 14 to the RC 16 or CP 18 via the back telemetry circuitry 78 and coil 80. The RC 16 or the CP 18 may then perform the necessary processing and control functions to adjust the stimulation parameters and transmit the adjusted set of stimulation parameters to the IPG 14 or otherwise control the IPG 14, so that the IPG 14 can generate the electrical stimulation energy in accordance with the adjusted set of stimulation parameters.

The IPG 14 further includes a rechargeable power source 82 and power circuits 84 for providing the operating power to the IPG 14. The rechargeable power source 82 may, e.g., include a lithium-ion or lithium-ion polymer battery. The rechargeable battery 82 provides an unregulated voltage to the power circuits 84. The power circuits 84, in turn, generate the various voltages 86, some of which are regulated and some of which are not, as needed by the various circuits located within the IPG 14. The rechargeable power source 82 is recharged using rectified AC power (or DC power converted from AC power through other means, e.g., efficient AC-to-DC converter circuits, also known as “inverter circuits”) received by the AC receiving coil 74. To recharge the power source 82, an external charger (not shown), which generates the AC magnetic field, is placed against, or otherwise adjacent, to the patient's skin over the implanted IPG 14. The AC magnetic field emitted by the external charger induces AC currents in the AC receiving coil 74. The charging and forward telemetry circuitry 76 rectifies the AC current to produce DC current, which is used to charge the power source 82. While the AC receiving coil 74 is described as being used for both wirelessly receiving communications (e.g., programming and control data) and charging energy from the external device, it should be appreciated that the AC receiving coil 74 can be arranged as a dedicated charging coil, while another coil, such as coil 80, can be used for bi-directional telemetry.

Additional details concerning the above-described and other IPGs may be found in U.S. Pat. No. 6,516,227, U.S. Patent Publication No. 2003/0139781, and U.S. Pat. No. 7,539,538, entitled “Low Power Loss Current Digital-to-Analog Converter Used in an Implantable Pulse Generator,” which are expressly incorporated herein by reference. It should be noted that rather than the IPG 14, the system 10 may alternatively utilize an implantable receiver-stimulator (not shown) connected to leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

Turning now to FIG. 5, an exemplary method 100 of performing an automated stimulation parameter adjustment in response to changes in distance between the one of the neurostimulation leads 12 to a target tissue site will be described. First, the SCS system 10 delivers electrical stimulation energy to a first set of the electrodes 26, in accordance with a set of stimulation parameters, thereby evoking a CAP in a population of neurons (step 102). The first set of electrodes 26 may be located on one or both of the neurostimulation leads 12. As previously mentioned, the CAP can be evoked by either the same electrodes that were used for delivering the stimulation energy to the target tissue site or other electrodes near the target tissue site.

Next, the SCS system 10 measures the magnitude of the eCAP at a second set of the electrodes 26 (step 104). The second set of electrodes 26 may be located on one or both of the neurostimulation leads 12. Preferably, the first set of electrodes 26 and the second set of electrodes 26 are carried by a neurostimulation lead implanted parallel to the axes of the neurons, so that it is ensured that the second set of electrodes 26 are in the proper location for measuring the eCAP travelling down the axes of the neurons. Alternatively, the second set of electrodes 26 may be carried by a separate lead that is placed orthogonally to the axes of the neurons, so that it is ensured that the second set of electrodes 26 are in the proper location for measuring the eCAP travelling down the axes of the neurons. As also previously mentioned, multiple CAPs may be evoked, measured, and averaged to increase the signal-to-noise ratio.

Next, the SCS system 10 compares the magnitude of the eCAP measurement to a threshold range including an upper threshold value and a lower threshold value. Based on this comparison, an electrical pulse parameter, such as pulse amplitude, pulse duration, and/or pulse rate can be adjusted (step 106).

In particular, if the magnitude of the measured eCAP indicates over-stimulation (i.e. when the measured magnitude is greater than the upper threshold value) (step 108), the SCS system 10 automatically adjusts the electrical pulse parameter in a manner that decreases the intensity level of the stimulation energy delivered to the target tissue site until the magnitude of the measured eCAP is within the threshold range (step 110). In general, decreasing the pulse amplitude, pulse duration, and pulse rate will decrease the intensity level of the delivered stimulation energy. If, at step 108, the magnitude of the measured eCAP indicates under-stimulation (i.e., when the measured magnitude is less than the lower threshold value) (step 112), the SCS system 10 adjusts the electrical pulse parameter in a manner that increases the intensity level of the stimulation energy delivered to the target tissue site until the magnitude of the measured eCAP is within the threshold range (step 114). In general, increasing the pulse amplitude, pulse duration, and pulse rate will increase the intensity level of the delivered stimulation energy. Notably, the electrical pulse parameter is adjusted until the magnitude of the magnitude of the measured eCAP is within the middle of the threshold range (e.g., a value equidistant from the upper and lower threshold values). In this manner, the automated stimulation parameter adjustment technique is more stable. That is, if the magnitude of the eCAP is maintained at the edges of the threshold range, the electrical pulse parameter will have to be constantly adjusted. If, at steps 108 and 112, the magnitude of the measured eCAP neither indicates over-stimulation or under-stimulation (i.e., when the measured magnitude is within the threshold range), the SCS system 10 returns to step 102.

The SCS system 10 may adjust the electrical pulse parameter by a step size (e.g., 0.1 mA for pulse amplitude, 10 μs for pulse duration, and 10 Hz for pulse rate), the amount of which may be determined based the desired resolution of adjustment. For example, a course resolution (large step size) may be selected in order to quicken the stimulation parameter adjustment technique, or a fine resolution (small step size) may be selected in order to improve the accuracy of the stimulation parameter adjustment technique. When the magnitude of the measured eCAP is greater than the upper threshold value, then the pulse amplitude, pulse duration, or pulse rate may be decreased by the step size, and when the magnitude of the measured eCAP is less than the lower threshold value, then the pulse amplitude, pulse duration, or pulse rate may be increased by the step size.

The electrical pulse parameter is incrementally increased or decreased until the magnitude of the measured eCAP is at the optimum value within the threshold range. That is, electrical stimulation energy is delivered to the first set of the electrodes 26 in accordance with the set of stimulation parameters (containing the modified electrical pulse parameter), thereby evoking a CAP in the population of neurons, the magnitude of the eCAP is measured at the second set of the electrodes 26, the magnitude of the measured eCAP is compared to the optimum value within the threshold range, the electrical pulse parameter is adjusted by the step size, and the process is repeated until the magnitude of the measured eCAP is at the optimum value within the threshold range.

Turning now to FIG. 6, another exemplary method 200 of performing an automated stimulation parameter adjustment in response to changes in distance between the one of the neurostimulation leads 12 to a target tissue site will be described. The method 200 differs from the method 100 described above, with the exception that the stimulation parameter that is adjusted is an electrode combination, rather than an electrical pulse parameter. Steps 202, 204, and 206, which are similar to the steps 102, 104, and 106 described above, are implemented by the SCS system 10 to evoke a CAP at the first set of electrodes, measure the eCAP at the second set of electrodes, and compare the magnitude of the eCAP measurement to the threshold range. Based on this comparison, the electrode combination is adjusted.

In particular, if, at step 208, the magnitude of the measured eCAP indicates over-stimulation (i.e. when the measured magnitude is greater than the upper threshold value), the SCS system 10, at step 210, adjusts the electrode combination in a manner that decreases the intensity level of the stimulation energy delivered to the target tissue site until the magnitude of the measured eCAP is within the threshold range. In general, adjusting the electrode combination, such that the resulting electrical field is displaced in a direction away from the target tissue site will decrease the intensity level of the stimulation energy delivered to the target tissue site. If, at step 208, the magnitude of the measured eCAP indicates under-stimulation (i.e., when the measured magnitude is less than the lower threshold value), the SCS system 10, at step 212, adjusts the electrode combination in a manner that increases the intensity level of the stimulation energy delivered to the target tissue site until the magnitude of the measured eCAP is within the threshold range. In general, adjusting the electrode combination, such that the resulting electrical field is displaced in a direction toward the target tissue site will increase the intensity level of the stimulation energy delivered to the target tissue site. As with the adjustment of the electrical pulse parameter described above, the electrode combination is adjusted until the magnitude of the measured eCAP is within the middle of the threshold range (e.g., a value equidistant from the upper and lower threshold values). If, at step 208, the magnitude of the measured eCAP neither indicates over-stimulation or under-stimulation (i.e., when the measured magnitude is within the threshold range), the SCS system 10 returns to step 202.

The SCS system 10 may adjust the electrode combination by a fractionalized step size (e.g., 5%, the amount of which may be determined based the desired resolution of adjustment. When the magnitude of the measured eCAP is greater than the upper threshold value, then the fractionalized electrode combination may be adjusted by the step size, such that the resulting electrical field is displaced away from the target tissue site, and when the magnitude of the measured eCAP is less than the lower threshold value, then the fractionalized electrode combination may be adjusted by the step size, such that the resulting electrical field is displayed toward the target tissue site. The fractionalized electrode combination is incrementally adjusted until the magnitude of the measured eCAP is at the optimum value within the threshold range. That is, electrical stimulation energy is delivered to the first set of the electrodes 26 in accordance with the set of stimulation parameters (containing the modified electrode combination), thereby evoking a CAP in the population of neurons, the magnitude of the eCAP is measured at the second set of the electrodes 26, the magnitude of the measured eCAP is compared to the optimum value within the threshold range, the fractionalized electrode combination is adjusted by the step size, and the process is repeated until the magnitude of the measured eCAP is at the optimum value within the threshold range.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A neurostimulation system, comprising:
a plurality of electrical terminals respectively configured for being electrically coupled to a plurality of electrodes implanted within a neural tissue;
stimulation output circuitry coupled to the plurality of electrical terminals;
monitoring circuitry coupled to the plurality of electrical terminals;
memory configured for storing a threshold value; and
controller/processor circuitry configured for:
prompting the stimulation output circuitry to deliver electrical stimulation energy to a first set of the electrodes in accordance with a stimulation parameter capable of treating a patient and evoking at least one compound action potential (CAP) in a population of neurons in the neural tissue;
prompting the monitoring circuitry to measure a magnitude of the at least one evoked CAP (eCAP) at a second set of the electrodes;
comparing a function of the at least one measured magnitude of the at least one eCAP to the threshold value; and
adjusting the stimulation parameter based on the comparison.

2. The neurostimulation system of claim 1, further comprising a stimulation lead carrying the first set of electrodes and the second set of electrodes.

3. The neurostimulation system of claim 1, wherein the at least one eCAP comprises a plurality of sequentially evoked CAPs.

4. The neurostimulation system of claim 3, wherein the function is an average of the plurality of eCAPs.

5. The neurostimulation system of claim 1, wherein the stimulation parameter is an electrical pulse parameter, and the controller/processor circuitry is configured for adjusting the electrical pulse parameter to vary an intensity level of the delivered electrical stimulation energy.

6. The neurostimulation system of claim 5, wherein the controller/processor circuitry is configured for increasing the electrical pulse parameter if the function of the at least one measured eCAP magnitude is below the threshold value.

7. The neurostimulation system of claim 5, wherein the controller/processor circuitry is configured for decreasing the electrical pulse parameter if the function of the at least one measured eCAP magnitude is above the threshold value.

8. The neurostimulation system of claim 5,
wherein the controller/processor circuitry is configured for comparing the function of the at least one measured eCAP magnitude to a first threshold value and a second threshold value greater than the first threshold value; and
wherein the controller/processor circuitry is configured for adjusting the electrical pulse parameter to increase the intensity level if the function of the at least one measured eCAP magnitude is below the first threshold value, adjusting the electrical pulse parameter to decrease the intensity level if the function of the at least one measured eCAP magnitude is above the second threshold value, and not adjusting the electrical pulse parameter if the function of the at least one eCAP magnitude is between the first threshold value and the second threshold value.

9. The neurostimulation system of claim 1, wherein the stimulation parameter is an electrode combination, and the controller/processor circuitry is configured for adjusting the electrode combination to displace a resulting electrical field relative to the neural tissue.

10. The neurostimulation system of claim 9, wherein the electrode combination is a fractionalized electrode combination.

11. The neurostimulation system of claim 1,
wherein controller/processor circuitry is configured for comparing the function of the at least one measured eCAP magnitude to first threshold value and a second threshold value greater than the first threshold value; and
wherein controller/processor circuitry is configured for adjusting the electrode combination to displace the resulting electrical field in a first direction if the function of the at least one measured eCAP magnitude is below the first threshold value, adjusting the electrode combination to displace the resulting electrical field in a second direction opposite to the first direction if the function of the at least one measured eCAP magnitude is above the second threshold value, and not adjusting the electrode combination if the function of the at least one eCAP magnitude is between the first threshold value and the second threshold value.

12. The neurostimulation system of claim 1, further comprising a biocompatible casing containing the plurality of electrical terminals, the stimulation output circuitry, the memory, the monitoring circuitry, and the controller/processor circuitry.

13. A method of treating a patient, comprising:
delivering electrical stimulation energy to a target tissue site in accordance with a stimulation parameter to treat the patient and evoke at least one compound action potential (CAP) in a population of neurons;
measuring a magnitude of the at least one evoked CAP (eCAP);
comparing a function of the at least one measured eCAP magnitude to a threshold value; and
adjusting the stimulation parameter based on the comparison.

14. The method of claim 13, wherein evoking the at least one CAP in the population of neurons comprises delivering electrical stimulation energy to a first set of electrodes, and measuring the at least one eCAP magnitude comprises measuring the at least one eCAP at a second set of electrodes.

15. The method of claim 14, wherein the first set of electrodes and the second set of electrodes are carried on a lead parallel to the population of neurons.

16. The method of claim 13, wherein the at least one eCAP comprises a plurality of sequentially evoked CAPs.

17. The method of claim 16, wherein the function is an average of the plurality of eCAPs.

18. The method of claim 13, wherein the threshold value is indicative of a therapeutic goal.

19. The method of claim 13, wherein the stimulation parameter is an electrical pulse parameter that is adjusted to vary an intensity level of the delivered electrical stimulation energy.

20. The method of claim 19, wherein the electrical pulse parameter is adjusted to increase the intensity level if the function of the at least one measured eCAP magnitude is below the threshold value.

21. The method of claim 19, wherein the electrical pulse parameter is adjusted to decrease the intensity level if the function of the at least one measured eCAP magnitude is above the threshold value.

22. The method of claim 19,
wherein the function of the at least one measured eCAP magnitude is compared to first threshold value and a second threshold value greater than the first threshold value; and
wherein the electrical pulse parameter is adjusted to increase the intensity level if the function of the at least one measured eCAP magnitude is below the first threshold value, is adjusted to decrease the intensity level if the function of the at least one measured eCAP magnitude is above the second threshold value, and is not adjusted if the function of the at least one eCAP magnitude is between the first threshold value and the second threshold value.

23. The method of claim 13, wherein the stimulation parameter is an electrode combination that is adjusted to displace a resulting electrical field relative to the target tissue site.

24. The method of claim 23, wherein the electrode combination is a fractionalized electrode combination.

25. The method of claim 23, wherein the function of the at least one measured eCAP magnitude is compared to first threshold value and a second threshold value greater than the first threshold value; and
wherein the electrode combination is adjusted to displace the resulting electrical field in direction away from the target tissue site if the function of the at least one measured eCAP magnitude is below the first threshold value, is adjusted to displace the resulting electrical field in a direction toward the target tissue site if the function of the at least one measured eCAP magnitude is above the second threshold value, and is not adjusted if the function of the at least one eCAP magnitude is between the first threshold value and the second threshold value.

26. The method of claim 13, wherein the electrical stimulation energy is delivered from a lead implanted adjacent the target tissue site, and the lead migrates relative to the target tissue site prior to the delivery of the electrical stimulation energy to the target tissue site.
